(19) United States
(12) Patent Application Publication
(10) Pub. No.: US 2017/0168069 A1
(43) Pub. Date: Jun. 15, 2017

(54) Mammalian Dickkopf 3 (DKK3) as Urinary Marker for Chronic Kidney Diseases

(71) Applicants: Deutsches Krebsforschungszentrum Stiftung des Öffentlichen Rechts, Heidelberg (DE); Ruprecht-Karls-Universität Heidelberg, Heidelberg (DE)

(72) Inventors: Giuseppina FEDERICO, Heidelberg (DE); Stefan PORUBSKY, Heidelberg (DE); Bernd ARNOLD, Heidelberg (DE); Hermann-Josef GRÖNE, Heidelberg (DE); Michael MEISTER, Heidelberg (DE)

(21) Appl. No.: 15/300,194

(22) PCT Filed: Mar. 31, 2015
(86) PCT No.: PCT/EP2015/056975

§ 371 Date: Sep. 28, 2016

(30) Foreign Application Priority Data
Mar. 31, 2014 (EP) 14162932.9

(51) Int. Cl. G01N 33/68

(52) U.S. Cl. CPC G01N 33/6893; G01N 2800/52; G01N 2333/4703; G01N 2800/347

(57) ABSTRACT

The present invention relates to dkk3 as a marker for diagnosing chronic kidney disease and assessing the risk of disease progression, as well as to related methods and uses. In particular, the present invention relates to method for diagnosing chronic kidney disease, comprising the steps of a) obtaining a urine sample comprising early morning urine (EMU) from a mammalian, preferably a human, patient to be diagnosed, b) measuring the amount of Dickkopf 3 (DKK3) protein and/or adducts thereof in said sample, and c) concluding on a chronic kidney disease of said patient, wherein a higher amount of DKK3 protein and/or adducts thereof in said sample compared to a healthy patient is indicative for a chronic kidney disease.

The present invention relates to dkk3 as a marker for diagnosing chronic kidney disease and assessing the risk of disease progression, as well as to related methods and uses. In particular, the present invention relates to method for diagnosing chronic kidney disease, comprising the steps of a) obtaining a urine sample comprising early morning urine (EMU) from a mammalian, preferably a human, patient to be diagnosed, b) measuring the amount of Dickkopf 3 (DKK3) protein and/or adducts thereof in said sample, and c) concluding on a chronic kidney disease of said patient, wherein a higher amount of DKK3 protein and/or adducts thereof in said sample compared to a healthy patient is indicative for a chronic kidney disease.

### BACKGROUND OF THE PRESENT INVENTION

Chronic kidney disease (CKD), also known as chronic renal disease, causes a progressive and permanent loss of the kidney function. CKD must be well separated from acute kidney disease (AKD), as this type of kidney disease is reversible and usually involves a rapid function loss of kidneys within 48 hours.

Over the last decade, the incidence rate of CKD increased. A classification system was developed by the Kidney Disease Outcome Quality Initiative (K/DOQI), based on the glomerular filtration rate (GFR) of the kidney.

The classification categorizes the disease in five stages; from a slightly diminished function to the establishment of a kidney failure, and a reduction of the GFR activity to between 15% to 19.5% of the original GFR. After this, the patient is considered to suffer from end stage renal disease (ESRD).

One of the major known indicators for CKD is the steady increase of creatinine, the breakdown product of muscles, by decreased kidney function, when analyzing a biological sample. This concentration difference occurs because the affected kidney does no longer provide filtration of creatinine with the same efficiency. Because of the exponential relation between the level of creatinine and the loss of kidney function, this marker must be regarded as providing rather course results. In children, creatinine is furthermore insufficient because of the low muscle mass as present. In practice, one attempt to bypass the problems with creatinine commonly is the calculation of an “eGFR” (estimated glomerular filtration rate), nevertheless, this can not resolve the underlying issues.

Common protein urine tests measure the amount of proteins, such as albumin, found in a urine sample. After a urine sample is provided, it is tested. A dipstick made with a color-sensitive pad is used to tell the level of protein in the urine. Normally, although small amounts of protein are in urine, they are not detected when a routine dipstick test is performed. This is because the kidney is supposed to keep most proteins in the blood.

During the past decade, the application of gene expression profiling in cancer research has resulted in development of new therapeutic targets. In contrast to the success of gene expression profiling in oncology, several challenges have severely limited the application of genomic profiling in nonmalignant kidney diseases. First, tissue availability is limited because diagnostic kidney biopsies or nonmalignant nephrectomies are performed relatively infrequently. Second, the composition of kidney tissue cores is inherently heterogeneous contributing to sampling error which renders standardized, quantitative gene expression profiling across large series of kidney biopsies technically challenging.

Kurose et al. (in: Kurose et al. Decreased expression of REIC/Dkk-3 in human renal clear cell carcinoma. J Urol. 2004 March; 171(3):1314-8) describe REIC/Dkk-3 as a new molecular target for therapeutic measures against renal clear cell carcinoma.

Thus, one of the most important unmet needs in renal medicine is the identification and validation of markers for CKD and—optimally—the progression thereof that facilitate targeted treatment of those at high risk, while avoiding unnecessary treatment. Other objects of the present invention will become apparent to the person of skill when studying the specification of the present invention.

In a first aspect thereof, the object of the present invention is solved by providing a method for diagnosing chronic kidney disease, comprising the steps of a) obtaining a urine sample from a mammalian, preferably a human, patient to be diagnosed, b) measuring the amount of Dickkopf 3 (DKK3) protein and/or adducts thereof in said sample, and c) concluding on a chronic kidney disease of said patient, wherein a higher amount of DKK3 protein and/or adducts thereof in said sample compared to a healthy patient is indicative for a chronic kidney disease

The mammalian patient can be a rat, mouse, goat, rabbit, sheep, horse, monkey or human, preferred is a mouse, rat or human.

In the context of the present invention, an “adduct” of the DKK3 protein shall mean a degradation product of DKK3 in the sample that, nevertheless, can be detected/is detected in the context of the present method. Adducts can be formed due to a reduced stability of the protein to be detected (here: DKK3) at the temperatures as present during sampling, shipping, storage, and/or analysis. Adducts may be formed by protease activities as present in the biological sample as taken from the patient to be diagnosed. Furthermore, also covalent protein adducts formed after exposure to xenobiotics can be included in the analysis.

The DKK3 gene is transcribed into three different isoforms (NM_015881, 2650 bp, NM_013253, 2635 bp, and NM_001018057, 2587 bp). Two of them result from alternative use of first exon (i.e. exon 1a and exon 1b, although they are both non-coding). One more variant lacks exon 1. All the variants share exons 2 to 8, and code for a 350 aa functioning protein. As used herein, the term “DKK3” shall also include these isoforms.

The present inventors established two animal models, a mechanical and a toxic one, in which a kidney insufficiency was established. Both physiological conditions reflect human diseases, namely:
The UUO—model; this model is induced in mice by unilateral ureteral ligation (UUO) of the left ureter. The animals rapidly develop, within a few days, severe tubulo-interstitial renal fibrosis with an inflammatory cell infiltrate in the affected kidney. An obstruction of the ureter can be found also in mammals, such as human children and adults.
The adenine diet model (ADM); High-adenine feeding in mice results in the formation of crystals in the renal tubules, with subsequent tubular injury and inflammation, obstruction, and marked fibrosis. These pathophysiologies can also be found in mammals such as humans, both in children and adults.

For a further discussion of animal models, see, for example, Ellen Neven and Patrick C. D'Haese (Vascular Calcification in Chronic Renal Failure—What Have We Learned From Animal Studies? Circulation Research. 2011; 108: 249-264).

US 2012/0135882 relates to methods and compositions for diagnosing chronic kidney disease. US 2012/0135882 generally seeks to provide a diagnosis of chronic kidney disease based on “panels” (i.e. several) of biomarkers as identified using expression analysis (i.e. “predictive expression signatures”). One of the biomarkers as mentioned is DKK3, which is measured in a panel, and such measurement—allegedly—can be performed in a biological sample, such as blood, urine, saliva, phlegm, gastric juices, and the like). US 2012/0135882 does not perform an analysis using urine, and no problems with this analysis are mentioned. Instead, expression analysis on a chip based on kidney total RNA (examples 2-5), and histological samples were used (example 6).

Upon a more detail analysis of US 2012/0135882, the document does not contain any information that would qualify it as a promising springboard for developing an EMU-based test for DKK3 in the context of kidney diseases as follows.

a) US 2012/0135882 uses a plurality of markers, an indication that no individually effective markers were identified. US 2012/0135882 mentions “significant” expression changes, but this must not be confused with overall significance for the diagnosis.

b) US 2012/0135882 uses an animal model that has no human correlate. A transgenic animal is used that produces unnaturally high levels of TGFβ1 in the liver, thus flooding the body with said protein. This leads to scarring of the glomeruli in the kidney, a physiological reaction not found in any known human kidney disease.

c) Furthermore, US 2012/0135882 did not employ wild-type mice as background to induce kidney disease, and therefore the data as produced with the transgenic mice can even not be reliably extrapolated to the situation in the mouse.

d) The expression of DKK3 is described to be specific for podocytes. Podocytes are cells that cover the filter structure of the kidney, and thus only constitute a very small population of kidney cells, in particular when compared with the epithelium of the tubuli. There is no known human kidney disease where a specific expression of a protein from podocytes could be measured in the urine.

e) US 2012/0135882 seeks to correlate the results as obtained with the situation in the human, for example, in IgA nephropathy (example 6). Nevertheless, only histological mouse samples were tested with commercial or academic antibodies for DKK3 expression, as it appears that DKK3 could not be detected in human samples.

Preferred is the method according to the present invention, wherein said sample comprises early morning urine (EMU), or consists or consists essentially of EMU. When samples are used that were collected during the day (as is the case with the so-called “metabolic cage”), the amounts or concentrations of DKK3 as measured could not be sufficiently correlated with kidney function or morphologic kidney disease.

According to the present invention, “early morning urine” (EMU), sometime also called “first morning urine” shall mean a sample of the first pass urine of the day, either from a patient to be diagnosed or from a group of patients, such as, for example, a group of children (i.e. a pooled sample).

In the context of the experiments leading to the present invention, it was surprisingly found that DKK3 as found in urine, and preferably the EMU, can be a potent indicator for kidney insufficiency and in particular for tubular atrophy and interstitial fibrosis.

Thus, according to the present invention, the expression of DKK3 and its amount and/or concentration in the EMU shows a clear correlation with kidney disease, such as, for example, tubular atrophy and interstitial fibrosis in a mammalian, preferably a human, patient.

Using wild-type mice instead of transgenic mice as used in US 2012/0135882, the inventors could furthermore show that—under conditions of stress in the kidney—DKK3 is not expressed in the glomeruli, but in the epithelium of the tubules. These epithelia provide more than 90% of the cells in the kidney and thus have a direct contact to the urinary system.

Preferred is the method according to the present invention, wherein said patient to be diagnosed is a child or adolescent. In the context of the present invention, urine samples taken from a larger cohort of children with glomerular disease (GN) and primary tubular disease (NPHP)—corresponding to a nephronopthisis—were analyzed. A non-invasive detection of kidney disease, such as, for example, tubular atrophy and interstitial fibrosis, is more difficult to achieve in children than in adults.

For measuring and/or a detection of the mammalian such as human DKK3 and/or adducts thereof in the sample, standard methods for assessing protein expression can be used, such as, for example, a method selected from the group of mass spectrometry, chromatography, SDS gel electrophoresis, and antigen/antibody reactions, such as, for example, Western blots and/or Enzyme-Linked Immunosorbent Assay (ELISA). Preferred is ELISA.

For measuring/detecting DKK3 in urine samples, as explained herein, commercial available antibodies can be used in order to detect, preferred are the methods according to the present invention, wherein said antigen/antibody reaction comprises the use of monoclonal antibodies and/or fragments thereof (such as Fab or scFv, and the like) that are specific for mammalian, such as human, DKK3 and/or adducts thereof. If required, the person of skill will be readily able to generate and/or produce suitable antibodies, in particular monoclonal antibodies and/or fragments thereof (such as Fab or scFv, and the like) that are specific for mammalian, such as human, DKK3 and/or adducts thereof.

Preferred is the method according to the present invention, wherein said chronic kidney diseases are selected from tubular atrophy and interstitial fibrosis.

Kidney interstitial fibrosis (IF) can be defined as the accumulation of abnormal amounts of collagen and related molecules in the interstitium of the cortex, which serve as structural scaffolding. Renal tubular atrophy is an inevitable consequence of the chronic occlusion of the ureter.

Yet another aspect of the present invention then relates to a method for monitoring the progress of a chronic kidney disease, comprising the steps of a) obtaining a urine sample from a mammalian, preferably a human, patient to be monitored having a chronic kidney disease, b) measuring the amount of Dickkopf 3 (DKK3) protein and/or adducts thereof in said sample, and c) concluding on the progress of said chronic kidney disease of said patient, wherein a higher amount of DKK3 protein and/or adducts thereof in said sample compared to an earlier sample from said patient is indicative for a progressing chronic kidney disease. Preferred is a method according to the present invention, wherein said patient having a chronic kidney disease has been diagnosed using a method according to the present invention as above.

Preferred is the method for monitoring according to the present invention, wherein said sample comprises early morning urine (EMU), or consists or consists essentially of EMU, either from a patient to be diagnosed or from a group of patients, such as, for example, a group of children (i.e. a pooled sample). Preferred is the method for monitoring according to the present invention, wherein said patient to be diagnosed is a child or adolescent.

For measuring and/or a detection of the mammalian, such as human, DKK3 and/or adducts thereof in the sample, also in the context of the monitoring standard methods for assessing protein expression can be used, such as, for example, a method selected from the group of mass spectrometry, chromatography, SDS gel electrophoresis, and antigen/antibody reactions, such as, for example, Western blots and/or Enzyme-Linked Immunosorbent Assay (ELISA). Preferred is ELISA.

Preferred is a method for monitoring according to the present invention, wherein said patient is undergoing treatment for a chronic kidney disease, such as, for example, tubular atrophy and/or interstitial fibrosis. Said treatment can preferably be performed as described below.

In addition to the above approaches, new targets for the therapy of chronic kidney diseases are desired. Thus, according to another aspect thereof, the present invention provides a method for detecting and/or identifying a compound suitable for the treatment of chronic kidney diseases, comprising the steps of a) administering a candidate compound to a mammal having a chronic kidney disease, and b) obtaining a urine sample from said mammal, c) measuring the amount of Dickkopf 3 (DKK3) protein and/or adducts thereof in said sample, and d) detecting and/or identifying a compound suitable for the treatment of said chronic kidney disease in said mammal, wherein a lower amount of DKK3 protein and/or adducts thereof in said sample compared to an earlier sample from said mammal is indicative for a compound suitable for the treatment of said chronic kidney disease. Said mammal can be a rat, mouse, goat, rabbit, sheep, horse, monkey or human, preferred is a mouse, rat or human.

Preferred is the method for detecting and/or identifying according to the present invention, wherein said sample comprises early morning urine (EMU), or consists or consists essentially of EMU, either from a patient to be diagnosed or from a group of patients, such as, for example, a group of children (i.e. a pooled sample). Preferred is the method for detecting and/or identifying according to the present invention, wherein said patient to be treated and/or diagnosed is a child or adolescent.

For measuring and/or a detection of the mammalian, such as human, DKK3 and/or adducts thereof in the sample, also in the context of the detecting and/or identifying standard methods for assessing protein expression can be used, such as, for example, a method selected from the group of mass spectrometry, chromatography, SDS gel electrophoresis, and antigen/antibody reactions, such as, for example, Western blots and/or Enzyme-Linked Immunosorbent Assay (ELISA). Preferred is ELISA.

More preferred is a method for detecting and/or identifying according to the present invention, wherein said compound is selected from the group consisting of a peptide library, a combinatory library, a cell extract, in particular a plant cell extract, a “small molecular drug”, an antisense oligonucleotide, an siRNA, an mRNA and an antibody or fragment thereof (such as Fab or scFv, and the like).

The method according to the present invention as described herein is thus suitable for the identification of compounds that can modulate the expression of DKK3 in a cell/in cells.

Preferred is a method for detecting and/or identifying according to the present invention, further comprising testing said compound(s) as detected/identified for its activity on chronic kidney diseases. Respective assays are known to the person of skill, and can be taken from the respective literature.

Preferred is a method for detecting and/or identifying according to the present invention, wherein steps a) to d) are repeated, and, optionally, chemically modifying said compound before said repeating. The thus identified candidate compound can then, in a preferred embodiment, modified in a further step. Modification can be effected by a variety of methods known in the art, which include, without limitation, the introduction of novel side chains or the exchange of functional groups like, for example, introduction of halogens, in particular F, Cl or Br, the introduction of lower alkyl groups, preferably having one to five carbon atoms like, for example, methyl, ethyl, n-propyl, isopropyl, n-butyl, isobutyl, tert-butyl, n-pentyl or isopentyl groups, lower alkenyl groups, preferably having two to five carbon atoms, lower alkynyl groups, preferably having two to five carbon atoms or through the introduction of, for example, a group selected from the group consisting of NH₂, NO₂, OH, SH, NH, CN, aryl, heteroaryl, COH or COOH group. The thus modified binding substances are than individually tested with a method of the present invention. If needed, the steps of selecting the candidate compound, modifying the compound, and testing compound can be repeated a third or any given number of times as required. The above described method is also termed “directed evolution” since it involves a multitude of steps including modification and selection, whereby binding compounds are selected in an “evolutionary” process optimizing its capabilities with respect to a particular property, e.g. its ability to modulate the expression of the dkk3 polypeptide.

Preferred is a method for detecting and/or identifying according to the present invention, wherein said patient is undergoing treatment for a chronic kidney disease, such as, for example, tubular atrophy and/or interstitial fibrosis. Said treatment can preferably be performed as described below.

Another aspect of the present invention relates to a method for manufacturing a pharmaceutical composition for treating or preventing chronic kidney disease, comprising the steps of: performing a method for detecting and/or identifying according to the present invention, and formulating said compound as detected and identified into a pharmaceutical composition.

In a further embodiment of the method of the present invention, the compound identified as outlined above, which may or may not have gone through additional rounds of modification and selection, is admixed with suitable auxiliary substances and/or additives. Such substances comprise pharmacological acceptable substances, which increase the stability, solubility, biocompatibility, or biological half-life of the interacting compound or comprise substances or materials, which have to be included for certain routes of application like, for example, intravenous solution, sprays, band-aids or pills.

Carriers, excipients and strategies to formulate a pharmaceutical composition, for example to be administered systemically or topically, by any conventional route, in particular enterally, e.g. orally, e.g. in the form of tablets or capsules, parenterally, e.g. in the form of injectable solutions or suspensions, topically, e.g. in the form of lotions, gels, ointments or creams, or in nasal or a suppository form are well known to the person of skill and described in the respective literature.

Administration of an agent, e.g., a compound can be accomplished by any method which allows the agent to reach the target cells. These methods include, e.g., injection, deposition, implantation, suppositories, oral ingestion, inhalation, topical administration, or any other method of administration where access to the target cells by the agent is obtained. Injections can be, e.g., intravenous, intradermal, subcutaneous, intramuscular or intraperitoneal. Implantation includes inserting implantable drug delivery systems, e.g., microspheres, hydrogels, polymeric reservoirs, cholesterol matrices, polymeric systems, e.g., matrix erosion and/or diffusion systems and non-polymeric systems, e.g., compressed, fused or partially fused pellets. Suppositories include glycerin suppositories. Oral ingestion doses can be enterically coated. Inhalation includes administering the agent with an aerosol in an inhalator, either alone or attached to a carrier that can be absorbed. The agent can be suspended in liquid, e.g., in dissolved or colloidal form. The liquid can be a solvent, partial solvent or non-solvent. In many cases, water or an organic liquid can be used.

Yet another aspect of the present invention is directed at a pharmaceutical composition for treating or preventing chronic kidney diseases, such as, for example, tubular atrophy and/or interstitial fibrosis, obtainable by a method according to the method as above.

In certain embodiments, the compound is administered to the subject by administering a recombinant nucleic acid, such as, for example, an anti-dkk3 RNA, for example an si-RNA. Preferably, the recombinant nucleic acid is a gene therapy vector.

Another aspect of the present invention relates to a method or use as described herein, wherein the pharmaceutical composition further comprises additional pharmaceutically active ingredients for treating chronic kidney diseases, such as, for example, tubular atrophy and/or interstitial fibrosis, i.e. chemotherapeutics.

Another aspect of the present invention then relates to a method for treating or preventing chronic kidney diseases, such as, for example, tubular atrophy and/or interstitial fibrosis in a mammalian, such as human, patient, comprising administering to said patient an effective amount of a pharmaceutical composition according to the invention as above. In general, the attending physician will base a treatment on the compound as identified, and optionally also on other individual patient data (clinical data, family history, DNA, etc.), and a treatment can also be performed based on the combination of these factors. This method of the present invention for example involves integrating individual diagnostic kidney disease data with patient clinical information and general healthcare statistics to enable, for example, the application of personalized medicine to the patient. Significant information about drug effectiveness, drug interactions, and other patient status conditions can be used, too.

Preferred is a therapeutic method according to the present invention, wherein said mammal to be treated is a rat, mouse, goat, rabbit, sheep, horse, monkey or human, preferred is a mouse, rat or human, such as a child, adolescent or adult. Treatment is meant to include, e.g., preventing, treating, reducing the symptoms of, or curing the disease or condition, i.e. chronic kidney diseases, such as, for example, tubular atrophy and/or interstitial fibrosis.

An “effective amount” is an amount of the compound(s) or the pharmaceutical composition as described herein that reduces on the expression and/or abundance of DKK3 in kidney cells and/or urine. The amount alleviates symptoms as found for chronic kidney diseases, such as, for example, tubular atrophy and/or interstitial fibrosis. Alleviating is meant to include, e.g., preventing, treating, reducing the symptoms of, or curing the disease (chronic kidney diseases, such as, for example, tubular atrophy and/or interstitial fibrosis) or condition.

The invention also includes a method for treating a subject at risk for chronic kidney diseases, such as, for example, tubular atrophy and/or interstitial fibrosis, and or a progression of these diseases, wherein a therapeutically effective amount of a compound as above is provided. Being at risk for the disease can result from, e.g., a family history of the disease, a genotype which predisposes to the disease, or phenotypic symptoms which predispose to the disease. A further aspect of the present invention is the use of a modulator of the expression of dkk3 for the manufacture of a pharmaceutical composition for treating or preventing chronic kidney diseases, such as, for example, tubular atrophy and/or interstitial fibrosis. Preferably, said modulator is an inhibitor of the expression of dkk3 in the kidney as described herein.

Yet another preferred aspect of the present invention then relates to the use of a diagnostic kit, comprising materials for diagnosing and/or monitoring chronic kidney disease in a human patient in a method according to the present invention as described herein, in one or separate containers, preferably comprising materials for measuring the amount of Dickkopf 3 (DKK3) protein and/or adducts thereof in a human urine sample, preferably comprising or consisting of early morning urine (EMU). Optionally, the kit comprises instructions for performing a method according to the present invention as described herein.

The kit may further comprise one or more of (iii) a buffer, (iv) a diluent, (v) a filter, (vi) a needle, or (v) a syringe. The container is preferably a bottle, a vial, a syringe or test tube; and it may be a multi-use container. The container may be formed from a variety of materials such as glass or plastic. Preferably the kit and/or container contains instructions on or associated with the container that indicates directions for reconstitution and/or use.

Preferred is the use according to the present invention, wherein said kit comprises materials for a method selected from the group of Western blots and/or Enzyme-Linked Immunosorbent Assay (ELISA). For example, the label may indicate that the lyophilized formulation is to be reconstituted to certain antibody concentrations as suitable for the above methods, such as ELISA.

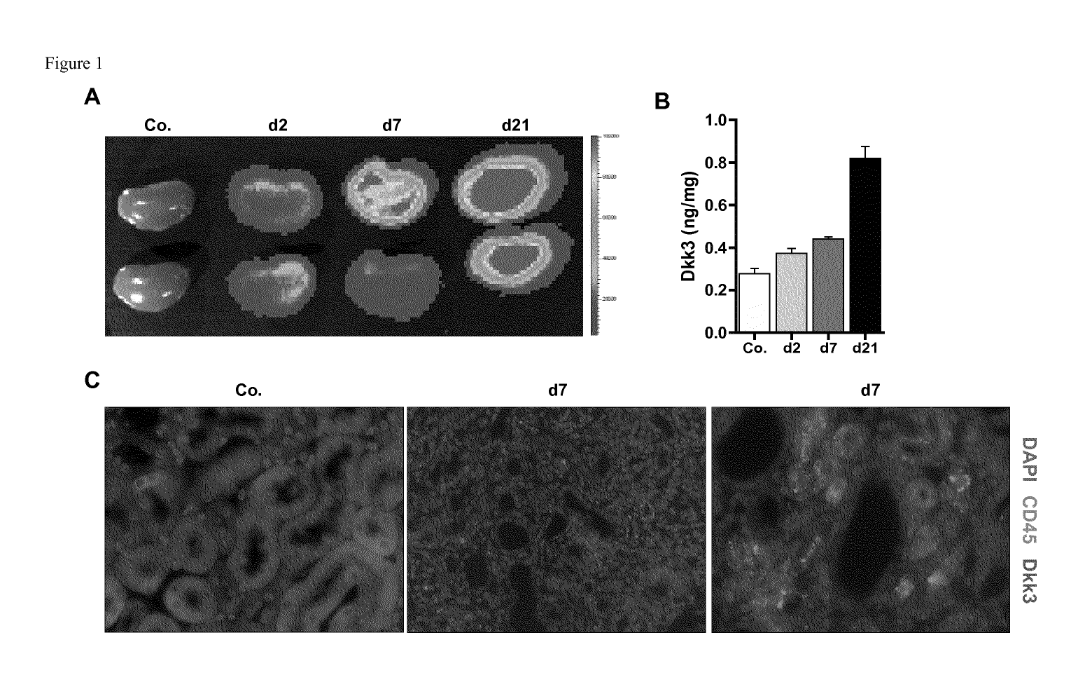

Further preferred is the use according to the present invention, wherein said kit comprises monoclonal antibodies or fragments thereof specific for human DKK3 and/or adducts thereof as described herein (see also FIG. 1).

The present invention shall now be further described in the following examples with reference to the accompanying Figures, nevertheless, without being limited thereto. All references as cited herein are incorporated by reference in their entireties. In the Figures,

FIG. 1 shows that renal Dkk3 production is induced in tubular epithelial cells during fibrosis development. (A+D) Ex vivo bioluminescence imaging of fibrotic kidneys of Dkk3-LCh mice (A) 2, 7, and 21 days after UUO induction. As a control, kidneys from untreated Dkk3-LCh mice were used. 5 min after i.p. injection of 150 mg/kg D-luciferin, mice were sacrificed and organs were taken out. After 5 min of incubation in a 1 mg/ml D-luciferin solution in PBS at 37° C. organs were imaged for 5 min. Colors display intensity of the emitted light (see scale). One representative experiment out of 3 is shown. (B+E) Amount of Dkk3 protein relative to organ weight in lysates of fibrotic kidneys (B) 2, 7 and 21 days after UUO induction or (E) 2, 7 and 28 days after starting of adenine feeding. As a control, kidneys from untreated mice were used. (C) Detection of mCherry expression in fibrotic kidneys of Dkk3-LCh mice 7 days after UUO induction. As a control kidneys of untreated Dkk3-LCh mice were used. Cryo sections from isolated kidneys were prepared and fluorochrome conjugated anti-mCherry staining (red) and anti-CD45 staining (green) was performed. Nuclei were counter stained with DAPI (blue). Shape and localization of mCherry positive cells identified them as tubular epithelial cells. One representative experiment out of three is shown.

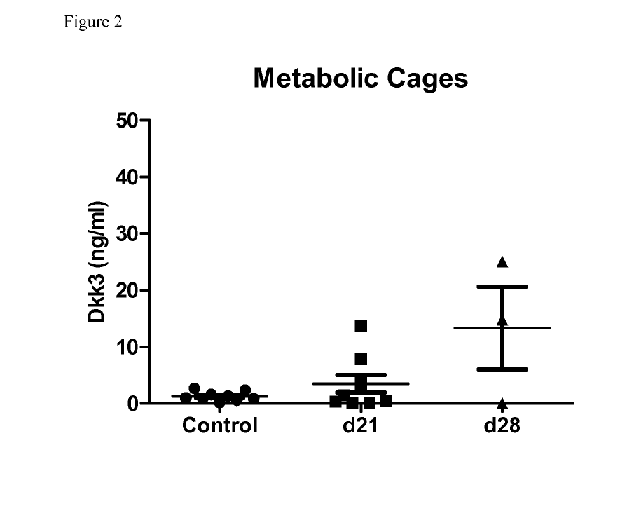

FIG. 2 shows the failure to reliably detect DKK3 with pooled urine samples (metabolic cages).

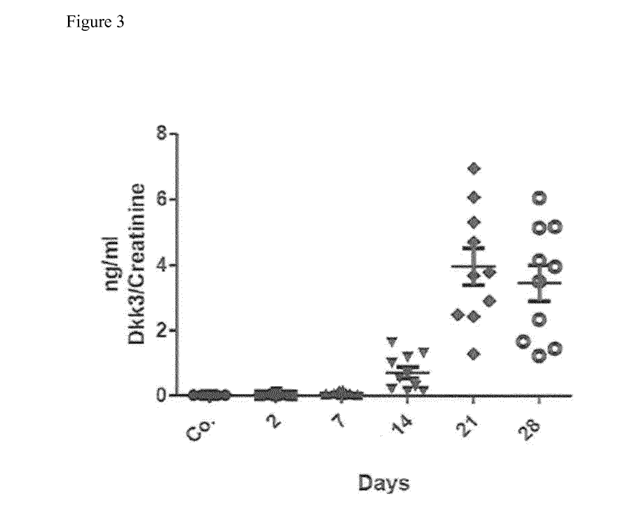

FIG. 3 shows that EMU was found to be suitable for a significant detection of DKK3; it shows the detection of DKK3 in the urine of mice in the model of the adenine-diet fed mouse. A clear increase of the quotient between creatinine and DKK3 was found, starting after 7 days.

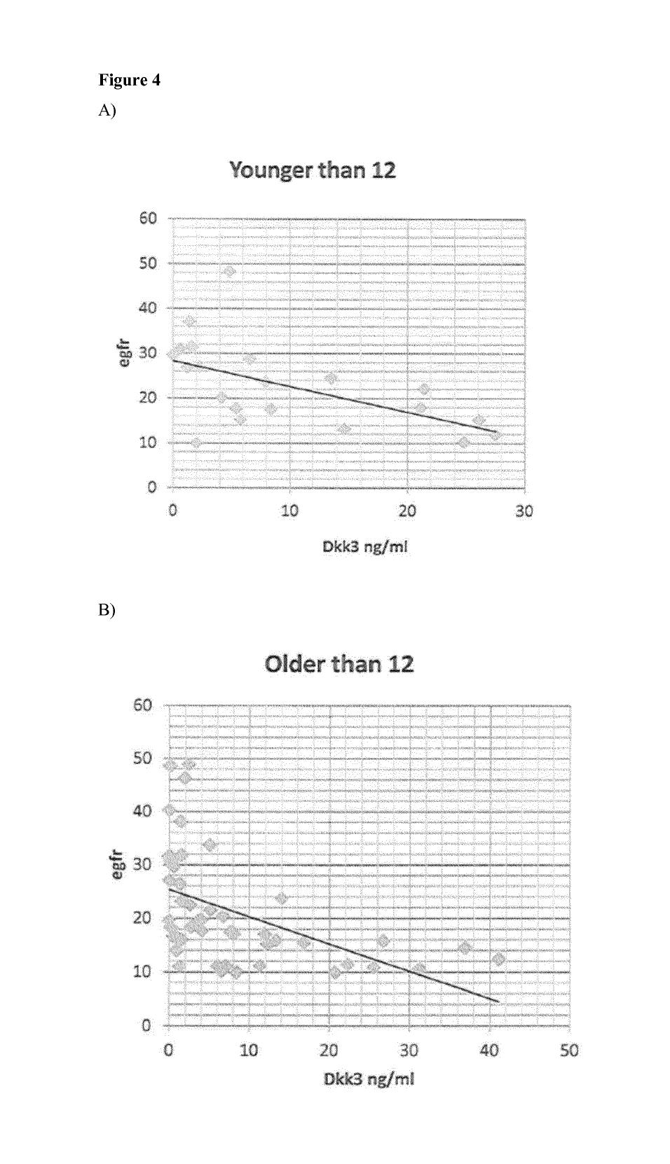

FIG. 4 shows measurements on EMU samples derived human from children having chronic kidney diseases, such as NPHP (A, see also example 3) versus samples from adults (B), showing an advantage of the assays according to the present invention for children. Egfr=estimated glomerular filtration rate; R² for younger than 12=0.3028; R² for older than 12=0.2399.

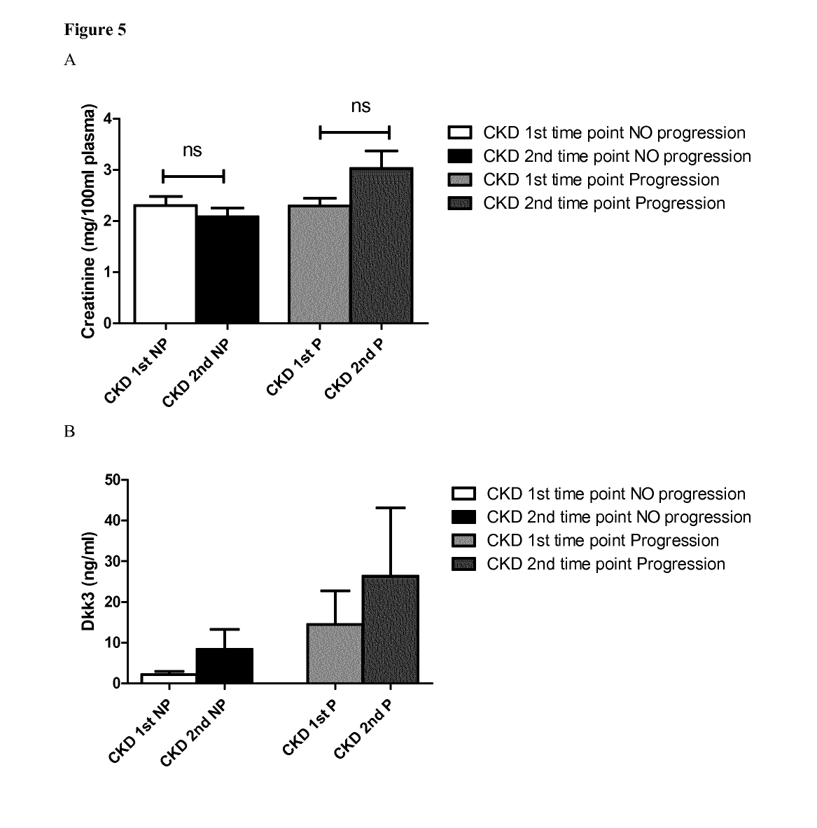

FIG. 5 shows A) the changes in the concentrations of creatinine and B) the changes in the concentrations DKK3 in urine between two successive measurements in progressive vs. non-progressive kidney disease.

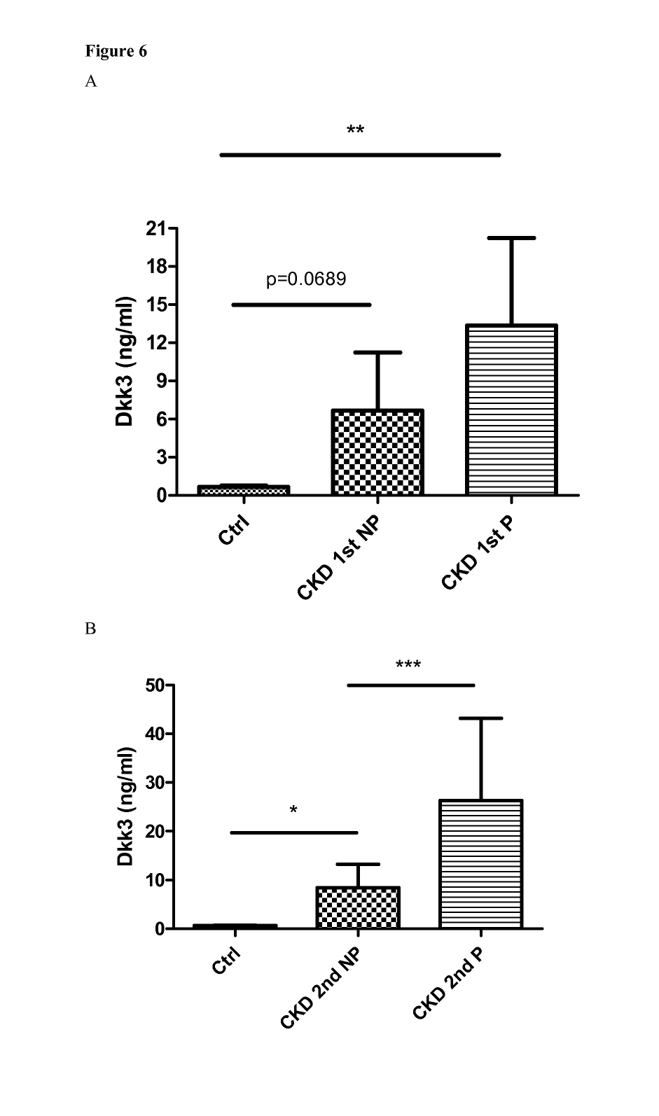

FIG. 6A shows the changes in the concentrations of DKK3 in samples collected from patients suffering from chronic kidney disease at a first time point. Ctrl=Control (Healthy kidneys); CKD 1^{st} NP=samples collected from patients suffering from chronic kidney disease (1^{st} time point). In these patients the disease did not progress; CKD 1^{st} P=samples collected from patients suffering from chronic kidney disease (1^{st} time point). In these patients the disease progressed. Ctrl; n=33; CKD 1^{st} NP; n=16; CKD 1^{st} P; n=16. FIG. 6B shows the changes in the concentrations of DKK3 in samples collected from patients suffering from chronic kidney disease at a second time point. Ctrl=Control (Healthy kidneys); CKD 2^{nd} NP=samples collected from patients suffering from chronic kidney disease (2^{nd} time point). In these patients the disease did not progress; CKD 2^{nd} P=samples collected from patients suffering from chronic kidney disease (2^{nd} time point). In these patients the disease progressed. Ctrl; n=33; CKD 2^{nd} NP; n=14; CKD 2^{nd} P; n=13.

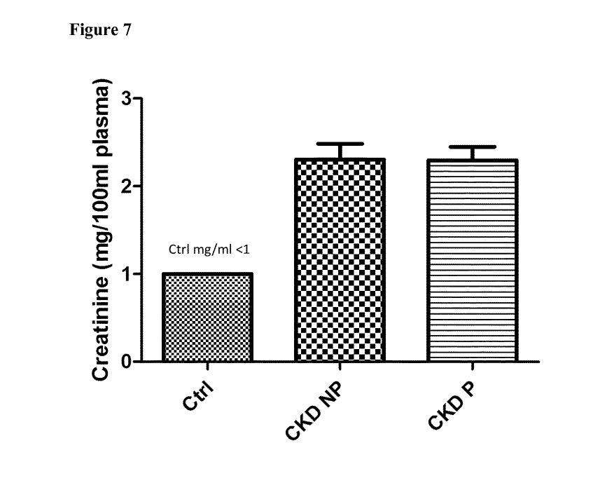

FIG. 7 shows that a creatinine concentration above 2 mg % equates to a Glomerular Filtration Rate (GFR) below 30-35 ml/min (normal value above 90 ml/min). Ctrl mg/ml<1→all control patients are in the normal range; a value above 1.2 mg % creatinine is abnormal. Ctrl=Control (Healthy kidneys); CKD NP=samples collected from patients suffering from chronic kidney disease. In these patients the disease did not progress; CKD P=samples collected from patients suffering from chronic kidney disease. In these patients the disease progressed.

### EXAMPLES

### Example 1: ELISA for the Detection of Human DKK3

For the analysis of human-Dkk3 protein, human EMU samples were tested using an ELISA based method. Micro Test III Flexible Assay Plate (Falcon #3912) was used as a sample plate.

Although other DKK3 Human ELISA Kits (e.g. from Creative Diagnostics, Shirley, N.Y., US; or from antibodies-online Inc., Atlanta, Ga., US) may be used, it was found that in many cases the specificity required adjustment to the conditions in urine samples.

### Buffers:

PBS pH 7.2

8.1 mM Na₂HPO₄×H₂O

1.5 mM K₂H₂PO₄

2.6 mM KCL

137 mM NaCl

PBS-Tween

PBS+0.05% Tween 20

Substrate Buffer pH 6.0

0.1 M KH₂PO₄

0.2% gelatin in PBS with 0.1% NaN₃

2 M H₂SO₄

Coating: Monoclonal antibody (recombinant human Dkk-3, CF, mAB11181; R&D Systems, Minneapolis, Minn., US)

Conjugate: Goat anti-human Dkk-3 mAB-Biotin-labelled (BAF1118; R&D Systems, Minneapolis, Minn., US); streptavidin peroxidase (Dianova, #016-030-084)

Substrate: Orthophenyldiamine (OPD, Sigma); H2O2 (30%) (Perhydrol, Merck)

Calibration protein: Recombinant human DKK-3 protein 0.1 mg/ml (1118-DK; R&D Systems, Minneapolis, Minn., US)

Equipment: Titertek 8-channel pipette
Eppendorf-pipettes and tips
Vaccu-Pette (96-channel pipette for microtiter plates, Neo-Lab)
Titertek Multiscan Plus MKII (ELISA photometer)

For coating, the plate was incubated over night at 4° C. with 100 μl MAB 11181 in a 1:500 dilution in PBS (final concentration μg/ml).

The plates were then washed by reversing the plates, and subsequent washing with about 200 μl of PBS-Tween. After the second washing step, it is important to remove as much liquid as possible.

For blocking, 200 μl of 0.2% gelatin in PBS is filled into each well, followed by incubation for 1 h at 37° C. The coated plates can be stored several months at 4° C. (drying has to be prevented).

In order to prepare a calibration curve, dilutions of the DKK3 calibration protein and the samples were prepared in PBS Tween. 100 μl were added into each well.

Calibration protein: huDKK3; serial dilution from 0.5-32 ng/ml

Test samples: four dilutions 1:50; 1:100; 1:500; and 1:1000

Incubation for 1 h at rt, followed by washing (4×) using PBS-Tween as above.

For the reaction with the first conjugate, 100 μl of goat-anti-human DKK3-biotin 1 μg/ml, diluted with PBS-Tween were added into each well, followed by incubation at rt for 1 h. Then, the plates were washed (4×) using PBS-Tween as above.

For the reaction with the second conjugate, 100 μl of streptavidin-peroxidase, diluted 1:1000 in PBS-Tween were added, again followed by washing (4×) using PBS-Tween as above.

A substrate reaction was obtained by weighing in of 11 mg of OPD, and complete dissolution of the OPD in substrate buffer (1 mg/ml). 11 μl H₂O₂ were added with subsequent shaking, and 100 μl of substrate solution were quickly added to each well. The plate was covered in order to protect the reactions from light. The reaction was stopped after about 1-10 minutes using 50 μl H₂SO₄ per well.

Finally, the reactions were measured using photometric analysis in a Multiscan Plus MKII photometer at OD₄₉₂. First, the calibration curve for huDKK3 was generated. All sample concentrations were determined with reference to said curve, and the actual protein concentration was calculated by multiplication with the respective dilution factor.

### Example 2: Expression Analysis of DKK3 in a Mouse Model

Using standard transgenic technology in mice, reporter mice were produced that contained transgenic dkk3-constructs, where the dkk3-promoter was linked to the marker protein GFP (provides a fluorescent signal when expressed). Since the physiology of the kidney in mice is comparable with the human kidneys, it is expected that the expression in mice is identical to the one in human. It was found that dkk3 was exclusively expressed in the epithelium of the tubuli (see FIG. 1).

### Example 3: Analysis of DKK3 in the EMU of Children

Using the above described assay, DKK3 was detected in the EMU of children having chronic kidney diseases. The results as depicted in the following table show a significant increase in chronic kidney disease.

| | | DKK3 |
|---|---|---|
| Sample | Disease | (ng/ml) |
| 18 | NSKD | 24.8 |
| 20 | NSKD | 7.9 |
| 38 | NSKD | 1.6 |
| 42 | NPHP | 7.6 |
| 44 | NPHP | 0.3 |
| 45 | GN | 1.5 |
| 48 | NSKD | 6.5 |
| 50 | NSKD | 6.7 |
| 60 | NSKD | 1.5 |
| 106 | NPHP | 0.2 |
| 114 | NPHP | 5.8 |
| 126 | NSKD | 22.3 |
| 137 | NSKD | 5.1 |
| 150 | NSKD | 25.5 |
| 153 | NPHP | 0.2 |
| 162 | NPHP | 0.5 |
| 167 | NSKD | 0.6 |
| 186 | NSKD | 41.1 |
| 217 | NSKD | 7 |
| 218 | NPHP | 2 |
| 262 | NPHP | 2.6 |
| 264 | NSKD | 0.6 |
| 266 | NSKD | 4.8 |
| 275 | NSKD | 1.3 |
| 309 | NSKD | 31.2 |
| 326 | NSKD | 0.1 |
| 331 | NPHP | 21.1 |
| 332 | NSKD | 36.9 |
| 334 | NSKD | 0.9 |
| 341 | NSKD | 3.7 |
| 342 | NSKD | 4.2 |
| 353 | NSKD | 14.6 |
| 361 | NSKD | 26.7 |
| 382 | NPHP | 13.5 |
| 384 | NSKD | 27.5 |
| 390 | NSKD | 26.1 |
| 397 | NSKD | 5 |
| 403 | NSKD | 6.6 |
| 412 | NSKD | 21.4 |
| 424 | NSKD | 1.9 |
| 430 | NPHP | 2.3 |
| 439 | NSKD | 8.4 |
| 509 | NSKD | 4 |
| 528 | NPHP | 1.2 |
| 558 | GN | 1.5 |
| 563 | GN | 11.3 |
| 568 | GN | 7.1 |
| 574 | NSKD | 20.7 |
| 576 | NSKD | 5.4 |
| 579 | NSKD | 16.8 |
| 634 | NSKD | 1.4 |
| 636 | NSKD | 1.3 |
| 654 | NSKD | 2.4 |
| 697 | NPHP | 2.7 |
| 702 | NPHP | 8.4 |
| 732 | NSKD | 12.3 |
| 736 | NSKD | 8.1 |
| 757 | NPHP | 6 |
| 874 | NSKD | 1.7 |
| 882 | NSKD | 0.9 |
| 933 | NSKD | 11.9 |
| 983 | NSKD | 13.3 |
| 1051 | GN | 1.4 |
| 1162 | NPHP | 4 |
| 1195 | GN | 0.1 |
| 1484 | NPHP | 14 |
| C1 | Age 10/w | 0.2 |
| C2 | Age 10/w | 0.1 |
| C3 | Age 15/m | 0.1 |
| C4 | Age 10/m | 0.1 |
| C5 | Age 6/w | 0.1 |
| C6 | Age 23/m | 0.1 |
| C7 | Age 22/m | 0.1 |

NSKD: Non specified chronic kidney disease;
GN: Glomerulonephritis;
NPHP: Nephronophthisis;
C1 to C7: controls with age and gender indicated

1. A method for diagnosing chronic kidney disease, comprising the steps of:
a) obtaining a urine sample from a mammalian patient to be diagnosed,
b) measuring the amount of Dickkopf 3 (DKK3) protein and/or adducts thereof in said sample, and
c) making a diagnosis of chronic kidney disease of said patient, wherein a higher amount of DKK3 protein and/or adducts thereof in said sample compared to a healthy patient is indicative of a chronic kidney disease.

2. The method according to claim 1, wherein said sample comprises early morning urine (EMU).

3. The method according to claim 1, wherein said measuring comprises a method selected from mass spectrometry, chromatography, SDS gel electrophoresis, and antigen/antibody reactions.

4. The method according to claim 1, wherein said chronic kidney disease is tubular atrophy or interstitial fibrosis.

5. The method according to claim 1, wherein the patient is a human child or adolescent.

6. A method for monitoring the progress of a chronic kidney disease, comprising the steps of:
a) obtaining a urine sample from a mammalian patient to be monitored having a chronic kidney disease,
b) measuring the amount of Dickkopf 3 (DKK3) protein and/or adducts thereof in said sample, and
c) monitoring the progress of said chronic kidney disease of said patient, wherein a higher amount of DKK3 protein and/or adducts thereof in said sample compared to an earlier sample from the patient is indicative of a progressing chronic kidney disease.

7. The method according to claim 6, wherein the patient having a chronic kidney disease has been diagnosed using a method comprising the steps of:
a) obtaining a urine sample from a patient to be diagnosed,
b) measuring the amount of Dickkopf 3 (DKK3) protein and/or adducts thereof in said sample, and
c) making a diagnosis of chronic kidney disease of said patient, wherein a higher amount of DKK3 protein and/or adducts thereof in said sample compared to a healthy patient is indicative of a chronic kidney disease.

8. The method according to claim 6, wherein said sample comprises early morning urine (EMU).

9. The method according to claim 6, wherein said measuring comprises a method selected from mass spectrometry, chromatography, SDS gel electrophoresis, and antigen/antibody reactions.

10. The method according to claim 6, wherein said patient is undergoing treatment for a chronic kidney disease.

11. A method for identifying a compound suitable for the treatment of a chronic kidney disease, comprising the steps of:
a) administering a candidate compound to a mammal having a chronic kidney disease,
b) obtaining a urine sample comprising early morning urine (EMU) from the mammal,
c) measuring the amount of Dickkopf 3 (DKK3) protein and/or adducts thereof in said sample, and
d) identifying a compound suitable for the treatment of said chronic kidney disease in the mammal, wherein a lower amount of DKK3 protein and/or adducts thereof in said sample compared to an earlier sample from said mammal is indicative of a compound suitable for the treatment of said chronic kidney disease.

12. The method according to claim 11, wherein steps a) to d) are repeated, and, optionally, chemically modifying said compound before said repeating.

13. A diagnostic kit comprising materials for measuring the amount of Dickkopf 3 (DKK3) protein and/or adducts thereof in a mammalian, urine sample comprising early morning urine (EMU) for diagnosing and/or monitoring chronic kidney disease in a patient.

14. The kit according to claim 13, wherein said kit comprises materials for a method selected from Western blots and/or Enzyme-Linked Immunosorbent Assay (ELISA).

15. A method for treating or preventing a chronic kidney disease in a human patient in need thereof, comprising administering to said patient an effective amount of a compound as identified according to claim 11.

16. The method according to claim 15, wherein said patient is a child or adolescent.

17. The method, according to claim 1, wherein the patient is a human.

18. The method, according to claim 2, wherein the sample consists of EMU.

19. The method, according to claim 6, wherein the patient is a human.

20. The method, according to claim 15, used to treat tubular atrophy and/or interstitial fibrosis.
